(19) United States
(12) Patent Application Publication
(10) Pub. No.: US 2019/0307596 A1
(43) Pub. Date: Oct. 10, 2019

(54) SUPPORT APPLIANCE FOR TESTICLES AND METHOD OF USE

(71) Applicant: GOLDA, INC. (an Ohio corporation), Beachwood, OH (US)

(72) Inventors: MARK WILLIAM CORRADO, Shaker Heights, OH (US); Donald Russell Burck, Orange Village, OH (US)

(73) Assignee: GOLDA, INC.

(21) Appl. No.: 15/949,618

(22) Filed: Apr. 10, 2018

(51) Int. Cl. A61F 5/40

(52) U.S. Cl. CPC A61F 5/40

(57) ABSTRACT

A therapeutic testicle support appliance and method of use is provided to assist the recovery of patients suffering from medical problems that have attendant swelling of the testicles and scrotum. The appliance has a support cradle with a pouch having legs extending from each of the ends of the pouch. These legs are adapted for adjustable interconnection with a belt worn by the patient around the waist and above the hips. The pouch itself is adjustable by means of draw-cord bungees and cord locks. Application and removal of the appliance is easily achieved by use of hook-and-loop fasteners accommodating secured engagement of the support cradle with the belt at any of various desired elevations.

### FIELD OF THE INVENTION

The invention herein resides in the art of medical appliances and, more particularly, to support devices such as those for limbs, ruptures, organs and the like. More specifically, the invention relates to a support appliance for testicles, giving support, aid and comfort to men suffering from illnesses that impact the genitals or testicles. More particularly, the invention relates to a medical appliance for testicles support adapted to cradle and support significantly enlarged testicles of men resulting from medical conditions to allow the men to be ambulatory with minimal or no testicular pain.

### BACKGROUND OF THE INVENTION

When men are stricken by certain maladies, their genitals or testicles are often affected. For example, liver diseases, kidney failure, heart transplant and the like typically require rehabilitation that focuses on getting the patient upright and walking. Problems often arise in this effort with fluid entering the testicles and scrotum, significantly adding to their size and weight, with the enlargement thereof actually blocking or inhibiting any fluid exit path from the testicles, aggravating the swelling.

Each testicle has a top tube and a bottom tube for the entry and exit of fluid. Both must remain open. However, the bottom tube may become pinched shut. When that happens, fluid buildup occurs, and the added weight of the testicles exacerbates the problem as the fluid continues to build up.

Not only does the enlargement and weight of the testicles cause excruciating pain to the patient when attempting to walk, but also that same weight continues to effect the closure of the bottom drain tube from the testicles, causing the enlargement and increased weight to continue.

In the past, hospitals have often employed bed sheets, pillow cases, and similar materials to fashion makeshift slings for holding or supporting the testicles while the patient is positioned upright and encouraged to walk. This sling or hammock allows the testicles and scrotum to drain by holding these structures high enough so that the drainage tubes are open and no longer pinched shut, thus reducing the swelling problem.

As is readily apparent, the makeshift appliances used in the past were neither reliable nor effective, but given to slipping, misalignment, and inadvertent removal, each of which would typically be attendant with pain, discomfort, and increased swelling.

There is a need in the art for a remedial appliance and its method of use to provide testicular support to accommodate drainage and facilitate mobility and exercise of recovering patients.

### SUMMARY OF THE INVENTION

The foregoing and other aspects of the invention which will become apparent as the detailed description proceeds are achieved by a therapeutic testicle support appliance, comprising a support cradle having a pouch, said pouch having first and second ends; a leg extending from each of said first and second ends of said pouch; and a belt having first and second end sections, said belt receiving and retaining said support cradle.

Other aspects of the invention are attained by the process for applying a therapeutic testicle support appliance to a patient, comprising attaching a support cradle to a belt, said support cradle having a pouch; wrapping said belt about the waist and above the hips of the patient, causing said support cradle to suspend from said belt; and placing the scrotum and testicles of the patient within said suspended cradle and adjusting an elevation of said cradle to provide support to the scrotum.

### BRIEF DESCRIPTION OF THE DRAWINGS

For a complete understanding of the various aspects, structures, and methods of the invention, reference should be made to the following detailed description and accompanying drawings wherein:

FIG. 1 is a perspective view of the support cradle of the therapeutic testicle support appliance of the invention;

FIG. 2 presents a first step of employing the appliance of the invention;

FIG. 3 presents the second step of the method of the invention;

FIG. 4 presents the third step in practicing the method of the invention;

FIG. 5 presents a fourth step of practicing the method of the invention;

FIG. 6 presents a fifth step of practicing the method of the invention; and

FIG. 7 is a perspective detailed view of the therapeutic testicle support appliance showing its method of adjustment.

### BEST MODE FOR CARRING OUT THE INVENTION

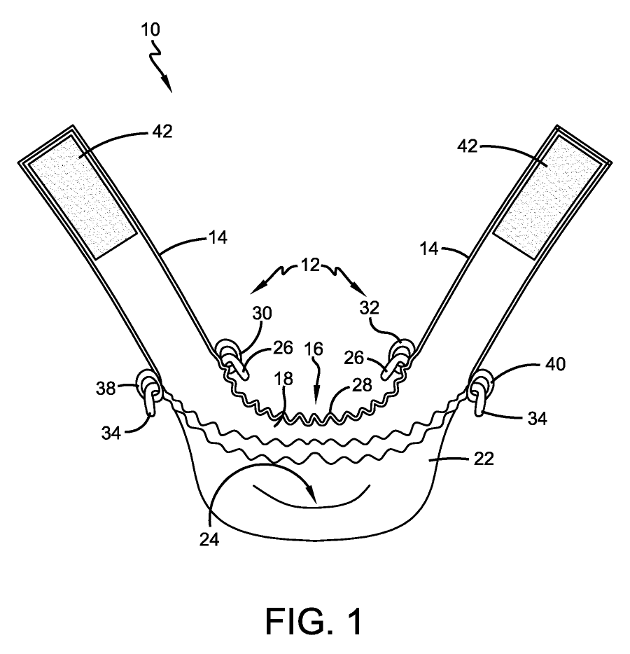

Referring now to the drawings and more particularly to FIG. 1, it can be seen that a support cradle made in accordance with the invention is designated generally by the numeral 10. The support cradle 10 is preferably formed by two pieces of cloth. The first piece of cloth forms a cradle 12 having a pair of legs 14 extending from opposite ends of a pouch 16, the pouch having sides 18 and 20. The second piece of cloth of the support cradle 10 comprises a pocket cover 22 attached to the side 20 of pouch 16 as by stitching or the like. An opening 24 provides access to a pocket defined between the pocket cover 22 and the pouch side 20, the opening 24 allowing access to the pocket for placement and retention of therapeutic materials such as an icepack or the like.

A draw-cord bungee 26 passes through a hemmed edge 28 of the side 18 of the pouch 16. The draw-cord bungee 26 is adjustably secured by cord locks 30, 32 at opposite ends of pouch 16. Similarly, a draw-cord bungee 34 passes through a hemmed edge 36 of the side 20 of pouch 16 and is adjustably secured by cord locks 38, 40. Those skilled in the art will appreciate that the draw-cord bungees 26, 34 along the opposed top edges of the pouch 16 and in selective locking engagement with respective cord locks 30, 32 and 38, 40 allow for adjustment of the physical size and comfort of the pouch 16. This allows the support cradle 10 to be adjusted to accommodate testicles of various sizes resulting from particular ailments. It is known in the art that the pouch 16 may be of a “small” size to accommodate testicles of the size of grapefruit, a “medium” size on the order of cantaloupes, and a “large” size on the order of honeydew melons, for lack of a more precise definition.

At the ends of each of the legs 14 is a patch of Velcro-type hook-and-loop material. According to the preferred embodiment of the invention, the pads 42 comprise the hook portions of hook-and-loop closures. As will become apparent hereinbelow, these hook patches are adapted to securedly interconnect with a loop material forming a support belt.

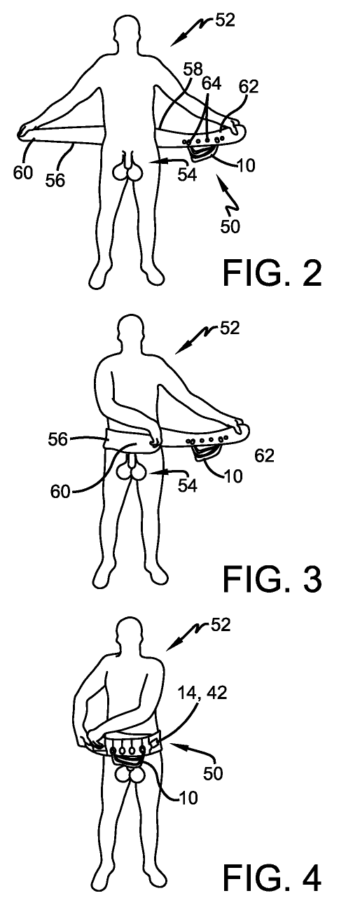

With reference now to FIGS. 2-7, and beginning with FIG. 2, an appreciation of the structure and use of the therapeutic testicle support appliance 50 of the invention may be obtained. Here, a patient 52 with an enlarged, swollen scrotum and associated testicles 54 is shown as donning the appliance 50 of the invention. A belt 56, which is preferably non-elastic, is tapered from a wide back section 58 to more narrow side or end sections 60, 62. Spaced apertures 64 are provided through one side section 62 of the belt 56 for receiving the legs 14 of the support cradle 10. The various holes or apertures 64 accommodate various sizes of individuals and, to a degree, the effective width of the pouch 16. The belt 56 has an external surface, away from the body of the patient 52, which is of a “loop” nature in the context of a hook-and-loop closure. The belt 56 does not stretch and is bound with piping at the edges thereof so that it does not fray. The inner surface against the patient's body has a smooth finish.

It is contemplated that the belt 56 will be provided in at least two sizes, although additional sizes may be found to be necessary. A small size will typically have a length on the order of approximately 45 inches, while a large belt would be on the order of 70 inches in length. It is contemplated that the back or center portion of the belt will have a width of 5-8 inches, and most preferably on the order of 6 inches, while the ends will have a width of 4-6 inches, and most preferably 5 inches. As is apparent from the drawings, the belt tapers from the back to each end.

With reference to FIGS. 2-6, an appreciation can be obtained with respect to the method of the invention by which the therapeutic testicle support appliance 50 may be employed. As shown in FIG. 2, the patient 52 may secure the ends of the belt 56 in opposite hands with the belt passed behind the patient 52 and oriented about the waist and just above the hips. As shown, the support cradle 10 has already been temporarily affixed to the belt. In practice, this is achieved by passing the legs 14 through appropriately selected apertures 64.

As shown in FIG. 3, the patient 52 then wraps the side section 60 of the belt 56 about his waist with his right hand and then, with his left hand, brings the side section 62 over the side section 60 as shown in FIG. 4. The hook pads 42 at the ends of the legs 14 are then engaged with the loop outer surface of the belt 56 to temporarily secure the wrapped belt 56 about the waist of the patient 52, as shown in FIG. 4.

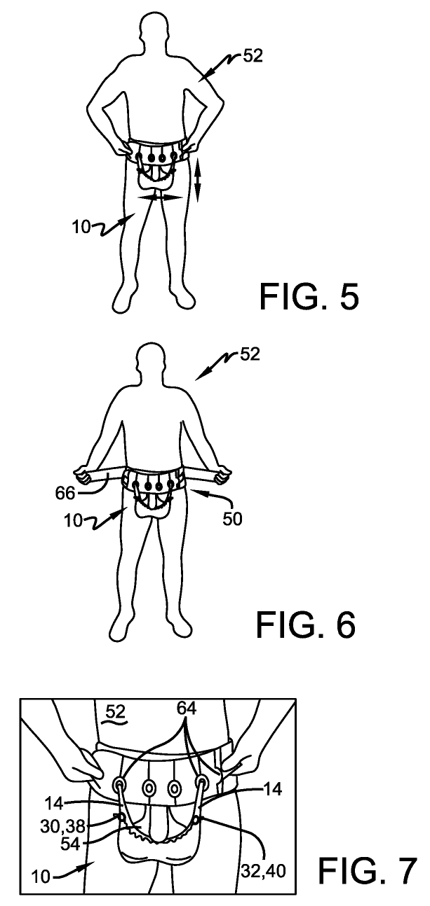

Next, as shown in FIGS. 5 and 7, the patient 52 places his scrotum 54 into the support cradle 10, adjusting it for size by means of the draw-cord bungees 26, 34 and associated cord locks 30, 32 and 38, 40.

Next, the patient may draw on the legs 14, pulling them through the associated apertures 64 to the elevation of desired comfort and support of the cradle 10, thereafter attaching the associated hook patches 42 to the outer loop surface of the belt 56 for supporting engagement of the support cradle 10. It is contemplated that the length of the leg 14 drawn by the right hand of the patient 52 will be of such length as to attach to the belt 56 beyond the side section 52 to effect an integral interconnection of the belt 56 with itself and with the cradle 10.

As shown in FIG. 6, an elastic band 66 may be fixed at the center back portion of the belt 56 and adapted to be drawn around and over the belt 56 to be finally secured in slightly forceful engagement with the belt 56 to further ensure that it is maintained about the waist and immediately above the hips of the patient 52. The elastic belt 66 may include hook-and-loop or other appropriate fasteners along its length and/or at its ends to ensure an adjustable, stable, and firm fit of the band 66 in its engagement with the belt 56.

It will be appreciated that the method and apparatus of the invention allows the patient to adjust the therapeutic testicle support appliance as necessary to meet his needs, assuring a comfort level that will give him the confidence to assume the mobility necessary for recovery from the malady causing the swelling. The appliance is given to ease of application and removal, as well as adjustment during use.

Thus it can be seen that the various aspects of the invention have been satisfied by the apparatus and method presented and described above. While in accordance with the patent statutes, only the best known and preferred embodiments of the invention have been presented and described in detail, the invention is not limited thereto or thereby. Accordingly, for an appreciation of the scope and breadth of the invention, reference should be made to the following claims.

What is claimed is:

1. A therapeutic testicle support appliance, comprising:
a support cradle having a pouch, said pouch having first and second end sections;
a leg extending from each of said first and second end sections of said pouch; and
a belt having first and second sections, said belt receiving and retaining said support cradle.

2. The therapeutic testicle support appliance as recited in claim 1, wherein said cradle is adjustable as to size and to a positional relationship with said belt.

3. The therapeutic testicle support appliance as recited in claim 2, wherein said pouch has draw-cord bungees on opposite sides thereof.

4. The therapeutic testicle support appliance as recited in claim 3, wherein said draw-cord bungees pass through associated cord locks.

5. The therapeutic testicle support appliance as recited in claim 4, wherein said pouch further comprises a pocket on a side thereof, said pocket being adapted to receive a cold pack.

6. The therapeutic testicle support appliance as recited in claim 2, wherein said belt has a plurality of apertures longitudinally spaced along said first end section thereof.

7. The therapeutic testicle support appliance as recited in claim 6, wherein an outer surface of said belt and end portions of said legs of said pouch are characterized by mating portions of a fastener.

8. The therapeutic testicle support appliance as recited in claim 7, wherein said fastener is a hook-and-loop fastener.

9. The therapeutic testicle support appliance as recited in claim 7, wherein said legs of said pouch pass through selected ones of said apertures and matingly fasten with said belt.

10. The therapeutic testicle support appliance as recited in claim 9, further comprising an elastic band securedly and circumferentially covering said belt.

11. A process for applying a therapeutic testicle support apparatus to a patient, comprising:
attaching a support cradle to a belt, said support cradle having a pouch;
wrapping said belt about the waist and above the hips of the patient, causing said support cradle to suspend from said belt; and
placing the scrotum and testicles of the patient within said suspended cradle and adjusting an elevation of said cradle to provide support to the scrotum.

12. The process as recited in claim 11, wherein said adjusting of an elevation of said cradle is undertaken by pulling legs at opposite ends of said cradle.

13. The process as recited in claim 12, wherein said legs are secured to said belt following adjustment.

14. The process as recited in claim 13, wherein said belt and said legs have mating hook-and-loop fastener portions thereon, the mating of said portions retaining said belt positioned on the patient and maintaining said elevation of said cradle.

15. The process as recited in claim 14, further comprising the step of adjusting a size of said pouch.

16. The process as recited in claim 15, wherein said adjustment of a size of said pouch is undertaken by draw-cord bungees by drawing said draw-cord bungees through associated cord locks.

17. The process as recited in claim 13, further comprising the step of securing an elastic band circumferentially about said belt.

18. The process as recited in claim 13, further comprising the step of placing a cooling pad within a pocket of said cradle adjacent pouch.
